(19) United States
(12) Patent Application Publication
(10) Pub. No.: US 2013/0096657 A1
(43) Pub. Date: Apr. 18, 2013

(54) PHOTOTHERAPY DEVICE

(71) Applicants: Yi-Wen YANG, Taipei City (TW); Jung-Chien Chang, Taipei City (TW); Ming-Da Yang, Taipei City (TW); Chen-Ho Cheng, Taipei City (TW); Yu-Chia Tsao, Taipei City (TW)

(72) Inventors: Yi-Wen YANG, Taipei City (TW); Jung-Chien Chang, Taipei City (TW); Ming-Da Yang, Taipei City (TW); Chen-Ho Cheng, Taipei City (TW); Yu-Chia Tsao, Taipei City (TW)

(73) Assignee: Forward Electronics Co., Ltd., Taipei City (TW)

(21) Appl. No.: 13/484,307

(22) Filed: May 31, 2012

(30) Foreign Application Priority Data
Oct. 13, 2011 (TW) 100137195

(51) Int. Cl. A61N 5/06

(52) U.S. Cl. 607/90

(57) ABSTRACT

The present invention relates to a phototherapy device, which is driven by a power supply and includes: a housing, having a top portion, a bottom portion and a handheld portion, where the top portion has a light outlet, the handheld portion connects the top portion and the bottom portion, and the handheld portion has at least one protruding part; a light-transmitting plate, which covers the light outlet; a light-blocking part, formed into a protruding rim along the circumferential direction of the light outlet; an LED module, disposed in the housing and corresponding to the light outlet; and a control module, electrically connected to the power supply and the LED module. Accordingly, the phototherapy device according to the present invention is suitable for handheld use.

### CROSS REFERENCE TO RELATED APPLICATION

This application claims the benefits of the Taiwan Patent Application Serial Number 100137195, filed on Oct. 13, 2011, the subject matter of which is incorporated herein by reference.

### BACKGROUND OF THE INVENTION

1. Field of the Invention

The present invention relates to a phototherapy device and, more particularly, to a phototherapy device suitable for handheld use.

2. Description of Related Art

With the improvement of the quality of the life, the cosmetology industry has developed quickly and phototherapy that can be used for treatment of acne, spot whitening, scar removal, wrinkle removal and whitening has become popular. A medical journal reported that propionibacterium acnes, which cause redness and inflammation associated with acne, contain porphyrin, and free radicals can be generated by reaction between blue light (its wavelength ranges from about 400 nm to 470 nm) and porphyrin to eradicate propionibacterium acnes so as to reduce redness and inflammation associated with acne. In addition, red light (its wavelength ranges from about 600 nm to 700 nm) is helpful for wound healing and anti-inflammation; yellow light (its wavelength ranges from about 550 nm to 600 nm) can improve the circulation of skin cells and promote the regeneration of skin cells; and green light (its wavelength ranges from about 500 nm to 550 nm) can be used to regulate the function of skin glands and oil secretion and to inhibit acne. Thereby, phototherapy can be performed by using light of a desired wavelength according to personal requirements to achieve a cosmetology or treatment object.

Currently, phototherapy mainly utilizes laser and pulsed light. However, laser and pulsed light instruments are expensive, require high operation technique and thus are not suitable for home use. Accordingly, ordinary light or LED light has been developed for phototherapy in place of the above-mentioned high intensity light. LEDs have advantages such as long lifetime and low power consumption. In particular, unlike laser or pulsed light that takes effect in destructive manners, LED light can achieve therapeutic effect through mild bio-stimulation. Thus, LED light has low danger and is more suitable for home use.

Therefore, the object of the present invention is to provide a phototherapy device suitable for home use.

### SUMMARY OF THE INVENTION

The object of the present invention is to provide a phototherapy device suitable for handheld use, which has enhanced operation safety.

To achieve the object, the present invention provides a phototherapy device, which is driven by a power supply and includes: a housing, having a top portion, a bottom portion opposite to the top portion and a handheld portion, where the top portion has a light outlet, the handheld portion connects the top portion and the bottom portion, and the handheld portion has at least one protruding part; a light-transmitting plate, which covers the light outlet; a light-blocking part, formed into a protruding rim along the circumferential direction of the light outlet; an LED module, disposed corresponding to the light outlet in an interior of the housing; and a control module, electrically connected to the power supply and the LED module. Herein, a drop from the top of the light-blocking part to the light-transmitting plate is created. That is, the light-blocking part is constructed as a protruding sidewall along the peripheral edge of the light-transmitting plate to achieve the effect of blocking light.

Therefore, users can hold the handheld portion of the phototherapy device according to the present invention during phototherapy, and the protruding part of the handheld portion can enhance convenience for handheld use. Besides, while using the phototherapy device of the present invention, the top of the light-blocking part of the phototherapy device can be placed in contact with a site to be treated so as to block light from reaching user's eyes or interfering in user's other works. Accordingly, operation safety and privacy can be enhanced.

The phototherapy device according to the present invention may connect to an outer power supply or use a battery as a power supply to drive the LED module. Herein, the battery may be a rechargeable battery, an ordinary battery or a micro battery. Preferably, the phototherapy device according to the present invention uses a battery as a power supply and thereby is advantageous to a portable design. Accordingly, in the present invention, the housing of the phototherapy device may have a power supply receiving part therein to receive the power supply.

In the phototherapy device of the present invention, the housing is preferably constructed by face-to-face assembling a first dish-shaped shell and a second dish-shaped shell, and the light outlet is formed at the first dish-shaped shell. Preferably, the first and second dish-shaped shells have a curve-shaped peripheral edge.

In the phototherapy device of the present invention, the housing is preferably made of a material with low light transmittance, such as materials with high reflectivity or high density, to reduce light leakage of the phototherapy device. Also, those skilled in the art can enhance fitness of assembly members of the phototherapy device through various structural designs to reduce light leakage beyond the light outlet.

In the phototherapy device of the present invention, the top portion and the bottom portion of the housing preferably are designed in a curve-shaped circumference and the handheld portion creates a curved outline along the circumferential direction of the top portion and the bottom portion of the housing. In addition, the handheld portion can create a convex curved surface from the top portion to the bottom portion, and preferably has plural protruding parts arranged along the circumferential direction of the top portion and the bottom portion. Accordingly, users can insert fingers between the protruding parts while holding the phototherapy device to enhance convenience for handheld use. In order to make the protruding parts conform to ergonomics, each of the protruding parts preferably has a convex curved surface, more preferably a convex curved surface and a concave curved surface linked to each other, along the circumferential direction of the top portion and the bottom portion. Therefore, the top portion, the bottom portion and the handheld portion of the phototherapy device according to the present invention make the housing have a body that conforms to ergonomics. Accordingly, in handheld use, discomfort can be reduced and smoothness can be enhanced.

In the phototherapy device of the present invention, the light-transmitting plate preferably is a diffuser lens that is advantageous to uniform illumination so as to protect user's eyes from being directly illuminated by phototherapeutic light and enhance the uniformity of the phototherapeutic effect.

The phototherapy device of the present invention may further include: a warning module electrically connected to the control module. The warning module can send out a warning signal upon the end of the therapeutic period to warn the user to stop phototherapy. The warning signal from the warning module is not particularly limited and can be anything that can achieve a warning effect, such as audio, sound or vibration.

The phototherapy device of the present invention may further include: an audio player module electrically connected to the control module. Users can enjoy music through the audio player module during the phototherapeutic period. Therefore, the low desire for using the device caused by users' feeling of a long and boring therapeutic period can be avoided and the user's interest in using the device can be enhanced.

In the phototherapy device of the present invention, the control module can include a logic circuit to control the action of the LED module, the warning module and the audio player module. The control module can be provided with one or more operation components and users can operate the above-mentioned modules through the operation components. For example, users can turn on/off the LED module and the audio player module through the operation components of the control module. Also, users can set the therapeutic period through the operation components to control the warning module to send out a warning signal upon the end of the therapeutic period. Preferably, the control module is provided with the operation components at locations corresponding to the handheld portion of the housing. Accordingly, users can place the phototherapy device at a site to be treated and then switch the device on by intuitive operation at the lateral surface of the device so as to prevent therapeutic light from directly illuminating user's eyes while starting the phototherapy device. Alternatively, the control module can be provided with a rotary operation component corresponding to the peripheral edge of the light outlet so that the device can be manipulated by rotation operation. Herein, the rotary operation component can function as the light-blocking part.

The phototherapy device of the present invention may further include: a sensor module electrically connected to the control module. The sensor module can detect whether the phototherapy device is located at a site to be treated and control the LED module to be on or off. Preferably, the sensor module is an IR sensor module. For example, when users place the phototherapy device within the sensing range of the sensor module, the sensor module would send a starting signal to the control module and then the LED module is driven through the control module. Alternatively, when the phototherapy device is placed beyond the sensing range of the sensor module, the LED module would be in a turn-off state.

In the phototherapy device of the present invention, the control module, the LED module, the warning module, the audio player module and the sensor module can all be received in the interior of the housing. The locations of the control module, the warning module and the audio player module are not particularly limited, while the LED module and the sensor module preferably are disposed corresponding to the light outlet, and more preferably corresponding to the center of the light outlet.

### BRIEF DESCRIPTION OF THE DRAWINGS

FIG. 1 shows a system diagram of a phototherapy device according to a preferred example of the present invention;

FIG. 2 shows an exploded view of a phototherapy device according to a preferred example of the present invention;

FIG. 3 shows a side view of a phototherapy device according to a preferred example of the present invention;

FIG. 4 shows a top view of a phototherapy device according to a preferred example of the present invention;

FIG. 5 shows a top view of a phototherapy device according to another preferred example of the present invention;

FIG. 6 shows a system diagram of a phototherapy device according to yet another preferred example of the present invention; and

FIG. 7 shows a top view of a phototherapy device according to yet another preferred example of the present invention.

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. It should be noted that these accompanying drawings are simplified. The quantity, shape and size of components shown in the drawings may be modified according to practically conditions, and the arrangement of components may be more complex. Other various aspects also may be practiced or applied in the invention, and various modifications and variations can be made without departing from the spirit of the invention based on various concepts and applications.

### EXAMPLE 1

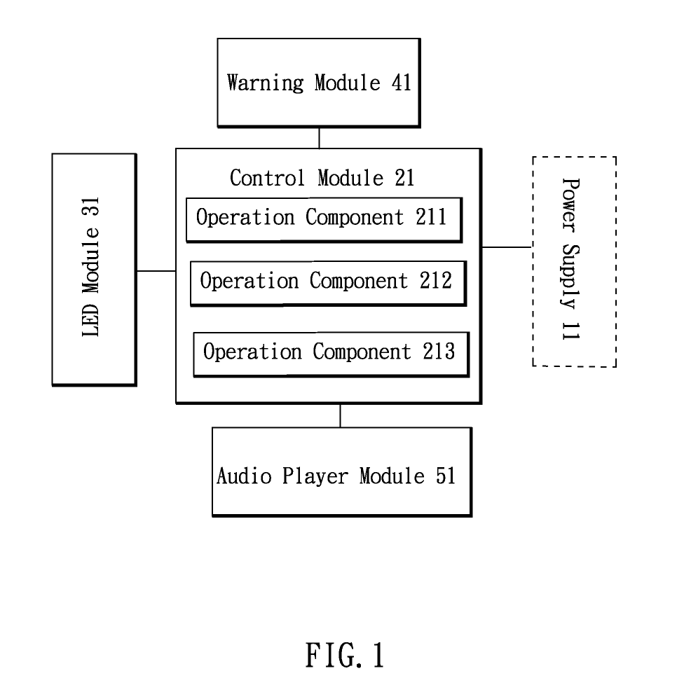

FIG. 1 shows a system diagram of a phototherapy device according to the present example. As shown in FIG. 1, the phototherapy device of the present example is driven by a power supply 11 and mainly includes a control module 21, an LED module 31, a warning module 41 and an audio player module 51 as action components. The control module 21 is electrically connected to the power supply 11, and the LED module 31, the warning module 41 and the audio player module 51 are electrically connected to the control module 21, respectively. The control module 21 has logic circuits for controlling the action of the LED module 31, the warning module 41 and the audio player module 51. For example, in the present example, users can control the LED module 31 to be on or off through the operation component 211 of the control module 21 and can set the phototherapeutic period through the operation component 212 to control the warning module 41 to send out warning signals (such as audio, sound or vibration) upon the end of the phototherapeutic period. In addition, users can also turn on/off the audio player module 51 through the operation component 213 so as to enjoy music during phototherapy and enhance their desire for using the device.

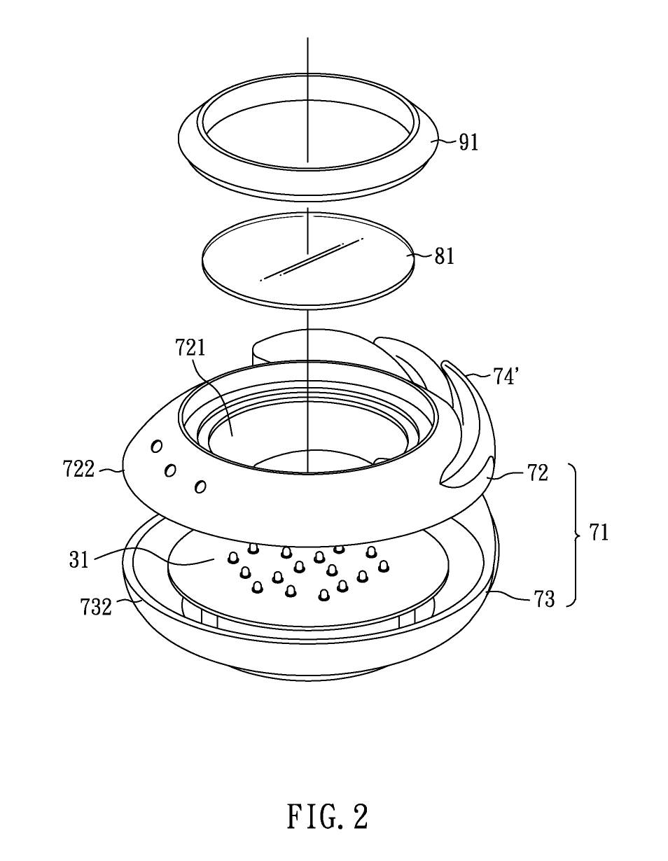

FIG. 2 shows an exploded view of the phototherapy device according to the present example. Those skilled in the art can dispose the above-mentioned control module, LED module, warning module and audio player module in the interior of the housing 71 in accordance with the connection relationship shown in FIG. 1. As shown in FIG. 2, the LED module 31 is disposed corresponding to the light outlet 721 of the housing 21 in the interior of the housing 71, and other action components (such as the control module, warning module and audio player module) are not limited to specific locations and thus not shown in the figure for brevity of drawings.

As shown in FIG. 2, in addition to the above-mentioned action components, the phototherapy device of the present example further includes: a housing 71, a light-transmitting plate 81 and a light-blocking part 91. The housing 71 is constructed by face-to-face assembling a first dish-shaped shell 72 and a second dish-shaped shell 73. The first dish-shaped shell 72 has a light outlet 721 so that therapeutic light from the LED module 31 can emit outwardly through the light outlet 721. As shown in FIG. 2, the first dish-shaped shell 72 and the second dish-shaped shell 73 have closed-curve shaped peripheral edges 722 and 732, respectively. In addition, plural protruding parts 74′ are formed at the peripheral edges 722 and 732 of the first dish-shaped shell 72 and the second dish-shaped shell 73 and are advantageous to handheld use of the device. The outline of the protruding parts 74′ at the first dish-shaped shell 72 is adapted to that at the second dish-shaped shell 73. Also, as shown in FIG. 2, the light-transmitting plate 81 covers the light outlet 721 and therapeutic light from the LED module 31 can pass through the light-transmitting plate 81 to reach a site to be treated. Herein, the light-transmitting plate 81 used in the present example is a diffuser advantageous to uniform illumination so that therapeutic light can uniformly illuminate a site to be treated and the user's eyes can be protected from being directly illuminated by the therapeutic light. Meanwhile, as shown in FIG. 2, the light-blocking part 91 is formed into a protruding rim along the circumferential direction of the light outlet 721 and thus the light-transmitting plate 81 is enclosed at its edge by a protruding sidewall. Accordingly, the light-blocking part 91 can block therapeutic light from illuminating the user's eyes and enhance privacy.

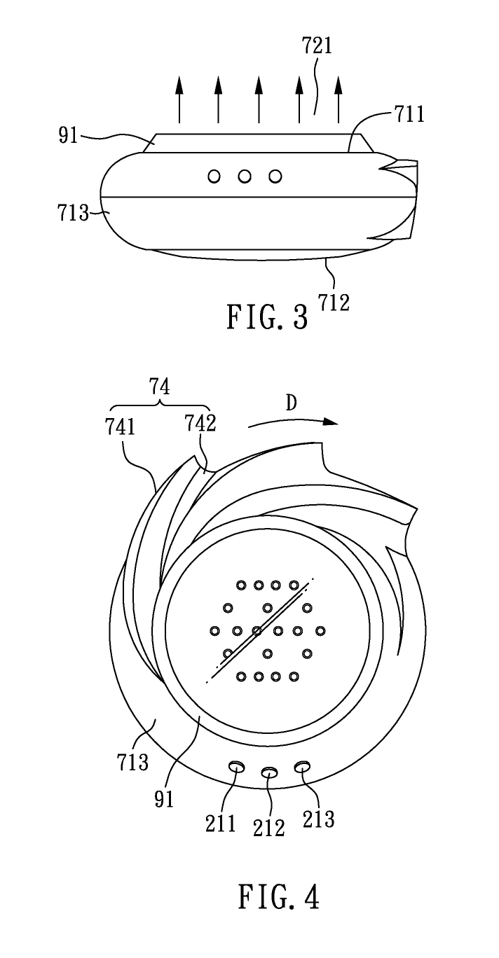

FIGS. 3 and 4 show a side view and a top view of the phototherapy device according to the present example, respectively.

As shown in FIG. 3, the housing after assembly has a top portion 711, a bottom portion 712 and a handheld portion 713. A light outlet 721 is provided at the top portion 711 so that therapeutic light can emit outwardly through the light outlet 721. Users can hold the handheld portion 713 that connects the top portion 711 and bottom portion 712 opposite to each other. As shown in FIG. 3, the handheld portion 713 creates a convex curve surface from the top portion 711 and the bottom portion 712, and the top portion 711 and the bottom portion 712 have a closed-curve shaped circumference (please also refer to FIGS. 2 and 4). Accordingly, the handheld portion 713 creates a close-curved outline along the circumferential direction of the top portion 711 and the bottom portion 712 (please also refer to FIG. 4). Moreover, as shown in FIG. 4, the handheld portion 713 of the phototherapy device has plural protruding parts 74 arranged along the circumferential direction D of the top and bottom portions. The protruding part 74 has a convex curved surface 741 and a concave curved surface 742 linked to each other along circumferential direction D of the top and bottom portions. Accordingly, the top portion 711, the bottom portion 712 and the handheld portion 713 of the housing construct a body that conforms to ergonomics so as to enhance smoothness in using the device. Additionally, as shown in FIG. 4, the control module is equipped with button-typed operation components 211, 212 and 213 at locations corresponding to the handheld portion 713. Accordingly, users can place the phototherapy device at a site to be treated and then switch the device on by intuitive push-button operation at the lateral surface of the device so that user's eyes can be protected from being directly illuminated by therapeutic light while starting the phototherapy device.

### EXAMPLE 2

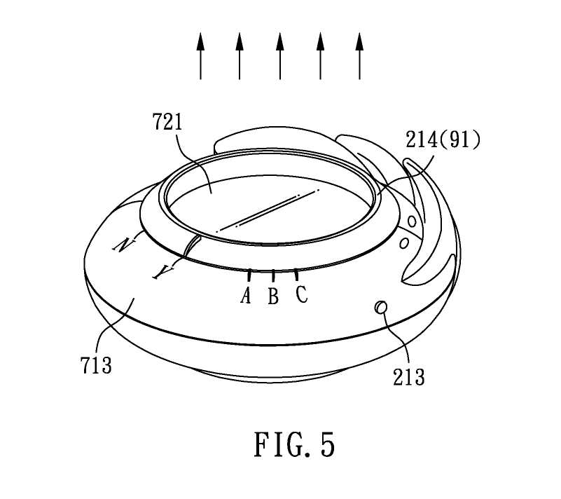

As shown in FIG. 5, the phototherapy device of the present example is the same as that illustrated in Example 1, except that the control module of the present example is provided with a rotary operation component 241 around the circumference of the light outlet 721. Users can control the LED module to be on or off and set phototherapeutic period through the rotary operation component 241. Meanwhile, the rotary operation component 241 can be used as a light-blocking part 91 to achieve the effect of blocking light. Additionally, the button-typed operation component 213 at the handheld portion 713 can be used to control the action of the audio player module. Specifically, when the operation component 214 is oriented to the label “N”, the LED module would be in a turn-off state. When operation component 214 is oriented to the label “Y”, the LED module could be driven and emit therapeutic light but the phototherapeutic period is not set. When operation component 214 is oriented to the time label “A”, “B” or “C”, the LED module would emit therapeutic light and the phototherapeutic period is set so that the warning module would send out a warning signal upon the end of the phototherapeutic period. Herein, the time labels “A”, “B” and “C” represent different phototherapeutic periods, respectively.

### EXAMPLE 3

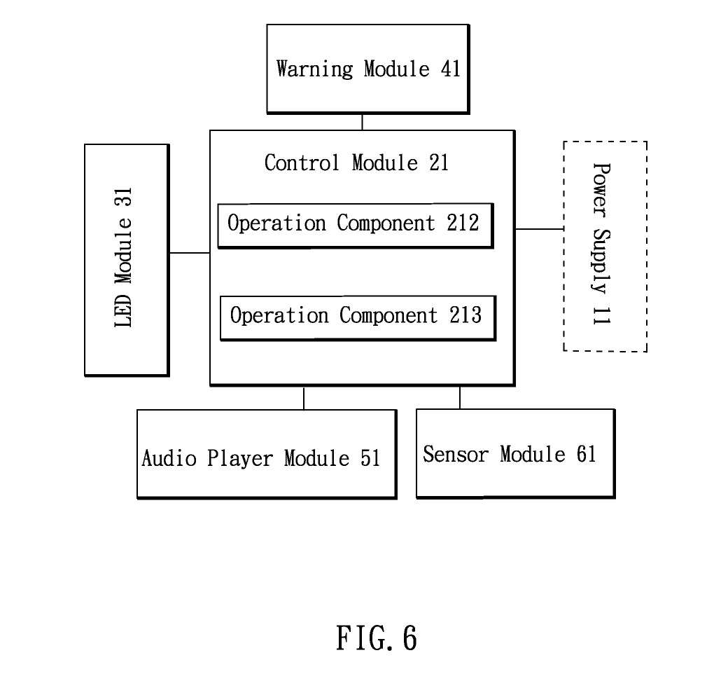

The phototherapy device of the present example is the same as that illustrated in Example 1, except that the phototherapy device of the present example is designed in an automatic start mode. As shown in FIG. 6, the phototherapy device of the present example mainly includes a control module 21, an LED module 31, a warning module 41, an audio player module 51 and a sensor module 61 as action components. The control module 21 is electrically connected to a power supply 11, and the LED module 31, the warning module 41, the audio player module 51 and the sensor module 61 are electrically connected to the control module 21, respectively. In the phototherapy device according to the present example, the sensor module 61 can detect whether the phototherapy device is placed at a site to be treated and then automatically control the LED module 31 to be on or off. Specifically, when users place the phototherapy device within a sensing range of the sensor module 61, the sensor module 61 would transmit a starting signal to the control module 21 and the LED module 31 can be driven by the control module 21. Alternatively, when the phototherapy device is placed beyond the sensing range of the sensor module 61, the LED module 31 would be in a turn-off state. Accordingly, the sensor module 61 can replace the operation component 211 of Example 1 to change the push-button start mode into the automatic start mode and thus enhance operation convenience. Herein, the present example uses an IR sensor module as the sensor module 61. Additionally, as mentioned in Example 1, users can set the therapeutic period and turn on/off the audio player model through the button-type operation components 212 and 213 regarding the phototherapy device of the present example.

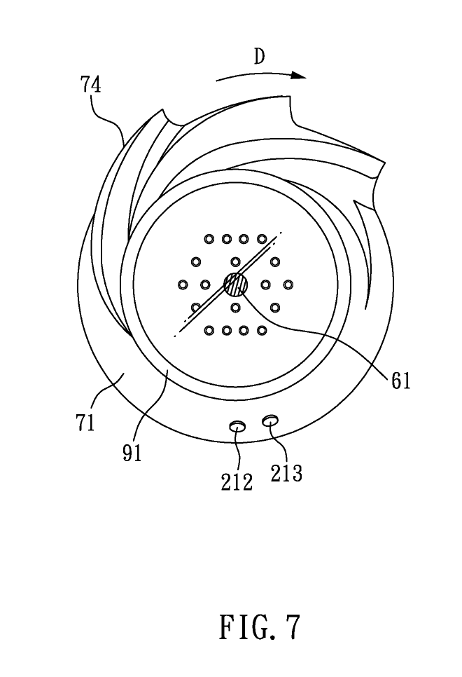

FIG. 7 shows a top view of the phototherapy device according to the present example. As shown in FIG. 7, the sensor module 61 is received in the interior of the housing 71 corresponding to the center of the light outlet.

The above examples are intended for illustrating the embodiments of the subject invention and the technical features thereof, but not for restricting the scope of protection of the subject invention. The scope of the subject invention is based on the claims as appended below.

What is claimed is:

1. A phototherapy device driven by a power supply, comprising:
a housing, having a top portion, a bottom portion opposite to the top portion and a handheld portion, wherein the top portion has a light outlet, the handheld portion connects the top portion and the bottom portion, and the handheld portion has at least one protruding part;
a light-transmitting plate, covering the light outlet;
a light-blocking part, formed into a protruding rim along a circumferential direction of the light outlet;
an LED module, disposed corresponding to the light outlet in an interior of the housing; and
a control module, electrically connected to the power supply and the LED module.

2. The phototherapy device of claim 1, wherein the top portion and the bottom portion have a curve-shaped circumference and the handheld portion creates a curved outline along a circumferential direction of the top portion and the bottom portion.

3. The phototherapy device of claim 1, wherein the handheld portion has a plurality of the protruding parts arranged along a circumferential direction of the top portion and the bottom portion.

4. The phototherapy device of claim 1, wherein the protruding part has a convex curved surface along a circumferential direction of the top portion and the bottom portion.

5. The phototherapy device of claim 1, wherein the protruding part has a convex curved surface and a concave curved surface linked to each other along a circumferential direction of the top portion and the bottom portion.

6. The phototherapy device of claim 1, wherein the handheld portion creates a convex curved surface from the top portion to the bottom portion.

7. The phototherapy device of claim 1, wherein the housing is constructed by face-to-face assembling a first dish-shaped shell and a second dish-shaped shell and the light outlet is formed at the first dish-shaped shell.

8. The phototherapy device of claim 1, wherein the light-transmitting plate is a diffuser lens.

9. The phototherapy device of claim 1, wherein the control module is provided with an operation component corresponding to the handheld portion of the housing.

10. The phototherapy device of claim 1, wherein the control module is provided with a rotary operation component around a peripheral edge of the light outlet and the rotary operation component is constructed as the light-blocking part.

11. The phototherapy device of claim 1, further comprising: a sensor module, electrically connected to the control module.

12. The phototherapy device of claim 1, wherein the sensor module is an IR sensor module.

13. The phototherapy device of claim 1, further comprising: a warning module, electrically connected to the control module.

14. The phototherapy device of claim 1, further comprising: an audio player module, electrically connected to the control module.
